(19) United States
(12) Patent Application Publication
(10) Pub. No.: US 2022/0133293 A1
(43) Pub. Date: May 5, 2022

(54) ORGAN RETRACTOR

(71) Applicants: Baxter International Inc., Deerfield, IL (US); Baxter Healthcare SA, Glattpark (Opfikon) (CN)

(72) Inventors: Sirisha Manam, Deerfield, IL (US); Krishnakumarsinh Parmar, Deerfield, IL (US); Elizabeth Dovec, Deerfield, IL (US); Olivia Enneking, Deerfield, IL (US); Tony Siebel, Deerfield, IL (US); Stephen Truesdell, Deerfield, IL (US); Maria Caterina Rizzoni, Deerfield, IL (US); Sophie Fain, Deerfield, IL (US); Samuel Ridgley, Deerfield, IL (US)

(21) Appl. No.: 17/514,495

(22) Filed: Oct. 29, 2021

Related U.S. Application Data
(60) Provisional application No. 63/107,832, filed on Oct. 30, 2020.

(51) Int. Cl. A61B 17/02; A61B 17/34

(52) U.S. Cl. CPC A61B 17/0218; A61B 2017/00818; A61B 17/3496; A61B 17/0206

(57) ABSTRACT

An organ retractor includes a plurality of retractor jaws, a central support structure, and a central housing. The central housing includes a threaded portion. The plurality of retractor jaws is hingedly connected to the central housing. The central support structure is coupled to the central housing. The plurality of retractor jaws is disposed in an open position by a spring.

### PRIORITY CLAIM

This application claims priority to U.S. Provisional Patent Application No. 63/107,832, filed Oct. 30, 2020, entitled “ORGAN RETRACTOR,” the entire contents of which are hereby incorporated in its entirety herein, including all tables, figures, and claims.

### BACKGROUND

Surgical procedures, such as gastrointestinal surgical procedures, often require access to unique locations within the body. For example, in a minimally invasive gastric-bypass procedure, the surgical devices are passed through a trocar and are manipulated (manually or remotely) to access the stomach. While clear access to the stomach is necessary to properly perform this procedure, unfortunately other organs and anatomical features may prevent, or at least make difficult, access to the stomach. For example, the liver's position in the body typically means that it is resting “on top” of the stomach when the patient is lying down during surgery. In this particular example, to access the stomach the liver must be retracted or held away from the stomach.

A typical organ retractor used in gastrointestinal surgical procedures is a Nathanson liver retractor. The Nathanson liver retractor requires a physical mount, such as a mount that is affixed to the patient's bed or a surgical stand, and a number of support arms for directional adjustment of a retractor. The retractor further includes a hook-like arm, which extends into the patient's body. This hook-like arm is used to retract the liver away from the surgical environment. However, this retraction device is disadvantageous for a number of reasons. The Nathanson liver retractor requires a number of components disposed outside the patient's body, such as the physical mount and the support arms. Each of these components must be sterilized prior to use. Further, each of these components requires setup and proper orientation in the operating room. The hook-like arm is typically a narrow metal hook, such as a 5 mm hook. To insert this hook-like arm into the patient, the surgeon must create a 5 mm incision on the patient's abdomen; the surgeon must also stitch up this incision after surgery. Additionally, the narrow hook-like arm puts unnecessary localized and individualized pressure onto the liver, during retraction. In some circumstances, this localized pressure is so severe as to cause trauma, such as hematoma, to the liver. Further, it is often difficult to remove the hook-like arm from the body, once the procedure is completed, given the hook-like arm's geometry.

Improved systems and methods for organ retraction are therefore needed.

### SUMMARY

To improve the retraction of liver, and other related organs, in the surgical environment, new retraction systems and methods are provided herein.

In light of the disclosure herein, and without limiting the scope of the invention in any way, in a first aspect of the present disclosure, which may be combined with any other aspect listed herein unless specified otherwise, an organ retractor includes a plurality of retractor jaws, a central support structure, and a central housing. The central housing includes a threaded portion. The plurality of retractor jaws is hingedly connected to the central housing. The central support structure is coupled to the central housing. The plurality of retractor jaws is disposed in an open position by a spring.

In a second aspect of the present disclosure, which may be combined with any other aspect listed herein unless specified otherwise, the central support structure is disposed between the plurality of retractor jaws.

In a third aspect of the present disclosure, which may be combined with any other aspect listed herein unless specified otherwise, the plurality of retractor jaws includes two retractor jaws.

In a fourth aspect of the present disclosure, which may be combined with any other aspect listed herein unless specified otherwise, the organ retractor further includes a threaded obturator, configured to engage with the threaded portion of the central housing.

In a fifth aspect of the present disclosure, which may be combined with any other aspect listed herein unless specified otherwise, responsive to engaging the threaded obturator with the threaded portion of the central housing, the organ retractor is capable of being manipulated via the threaded obturator.

In a sixth aspect of the present disclosure, which may be combined with any other aspect listed herein unless specified otherwise, manipulation includes lifting the threaded obturator, such that the organ retractor is lifted to support an organ.

In a seventh aspect of the present disclosure, which may be combined with any other aspect listed herein unless specified otherwise, the organ is generally supported by the plurality of retractor jaws and the central support structure.

In an eighth aspect of the present disclosure, which may be combined with any other aspect listed herein unless specified otherwise, responsive to lifting the threaded obturator, the threaded obturator is clamped at an exterior of a patient, such that the threaded obturator is prevented from sliding within an incision on the patient.

In a ninth aspect of the present disclosure, which may be combined with any other aspect listed herein unless specified otherwise, the central housing includes an end piece, configured to be manipulated by a surgical grasping tool.

In a tenth aspect of the present disclosure, which may be combined with any other aspect listed herein unless specified otherwise, the plurality of retractor jaws, the central support structure, and central housing are configured to be inserted, into a patient, via a port.

In an eleventh aspect of the present disclosure, which may be combined with any other aspect listed herein unless specified otherwise, the plurality of retractor jaws are inserted into the patient in a closed configuration, such that the plurality of retractor jaws are closed around the central support structure.

In a twelfth aspect of the present disclosure, which may be combined with any other aspect listed herein unless specified otherwise, responsive to exiting the port and entering the patient, the plurality of retractor jaws are disposed into the open position via the spring, away from the central support structure.

In a thirteenth aspect of the present disclosure, which may be combined with any other aspect listed herein unless specified otherwise, an organ retractor includes two retractor jaws, hingedly coupled to one another, and a central support structure. The central support structure is disposed between the retractor jaws. The retractor jaws are disposed in an open position by a spring.

In a fourteenth aspect of the present disclosure, which may be combined with any other aspect listed herein unless specified otherwise, the central support structure is sized and shaped to retract a liver.

In a fifteenth aspect of the present disclosure, which may be combined with any other aspect listed herein unless specified otherwise, the organ retractor further includes a central housing including a threaded portion, wherein the retractor jaws are hingedly connected to the central housing, and wherein the central support structure is coupled to the central housing.

In a sixteenth aspect of the present disclosure, which may be combined with any other aspect listed herein unless specified otherwise, the organ retractor further includes a threaded obturator, configured to engage with the threaded portion of the central housing.

In a seventeenth aspect of the present disclosure, which may be combined with any other aspect listed herein unless specified otherwise, responsive to engaging the threaded obturator with the threaded portion of the central housing, the organ retractor is capable of being manipulated via the threaded obturator.

In an eighteenth aspect of the present disclosure, which may be combined with any other aspect listed herein unless specified otherwise, manipulation includes lifting the threaded obturator, such that the organ retractor is lifted to support an organ.

In a nineteenth aspect of the present disclosure, which may be combined with any other aspect listed herein unless specified otherwise, responsive to lifting the threaded obturator, the threaded obturator is clamped at an exterior of a patient, such that the threaded obturator is prevented from sliding within an incision on the patient.

In a twentieth aspect of the present disclosure, which may be combined with any other aspect listed herein unless specified otherwise, the central housing includes an end piece, configured to be manipulated by a surgical grasping tool.

In a twenty-first aspect of the present disclosure, any of the structure, functionality, and alternatives discussed in connection with any of FIGS. 1 to 13 may be combined with any of the structure, functionality, and alternatives discussed in connection with any other one or more of FIGS. 1 to 13.

In light of the disclosure and aspects set forth herein, it is accordingly an advantage of the present disclosure to provide an organ retractor with simplified structure for support, improving ease of use and reducing the number of components needed.

It is another advantage of the present disclosure to provide an organ retractor with a reduced footprint, so as to utilize existing trocars and avoid additional large incisions.

It is a further advantage of the present disclosure to provide an organ retractor that retracts organs over a distributed surface, such as multiple arms and support structures, so as to reduce risk of trauma.

It is yet another advantage of the present disclosure to provide an organ retractor that is easy to manipulate within the patient's body cavity and is, likewise, easy to remove from the patient's body cavity.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been selected for readability and instructional purposes, and not to limit the scope of the inventive subject matter.

### BRIEF DESCRIPTION OF THE FIGURES

Understanding that figures depict only typical embodiments of the invention and are not to be considered to be limiting the scope of the present disclosure, the present disclosure is described and explained with additional specificity and detail through the use of the accompanying figures. The figures are listed below.

FIG. 1 illustrates an exploded perspective view of a retractor, according to an example embodiment of the present disclosure.

FIG. 2 illustrates side and exploded perspective views of a threaded obturator, according to an example embodiment of the present disclosure.

FIG. 3 illustrates exploded and perspective views of a clamp, according to example embodiments of the present disclosure.

FIG. 4 illustrates top views of retractor jaws, of varying sizes, according to example embodiments of the present disclosure.

FIG. 5 illustrates top views of retractor jaws, in open and closed configurations, according to example embodiments of the present disclosure.

FIGS. 6 to 7 illustrate retractor jaws, according to example embodiments of the present disclosure.

FIGS. 8 to 10 illustrate central housing and support structures, according to example embodiments of the present disclosure.

FIG. 11 illustrates various views of a torsion spring, according to an example embodiment of the present disclosure.

FIG. 12 illustrates various views of a spring dowel pin, according to an example embodiment of the present disclosure.

FIG. 13 illustrates various views of a jaw dowel pin, according to an example embodiment of the present disclosure.

### DETAILED DESCRIPTION

Certain embodiments described herein relate generally to the field of organ retraction. More particularly, some embodiments described herein relate to the retraction of organs, such as a liver, to improve access to the surgical environment.

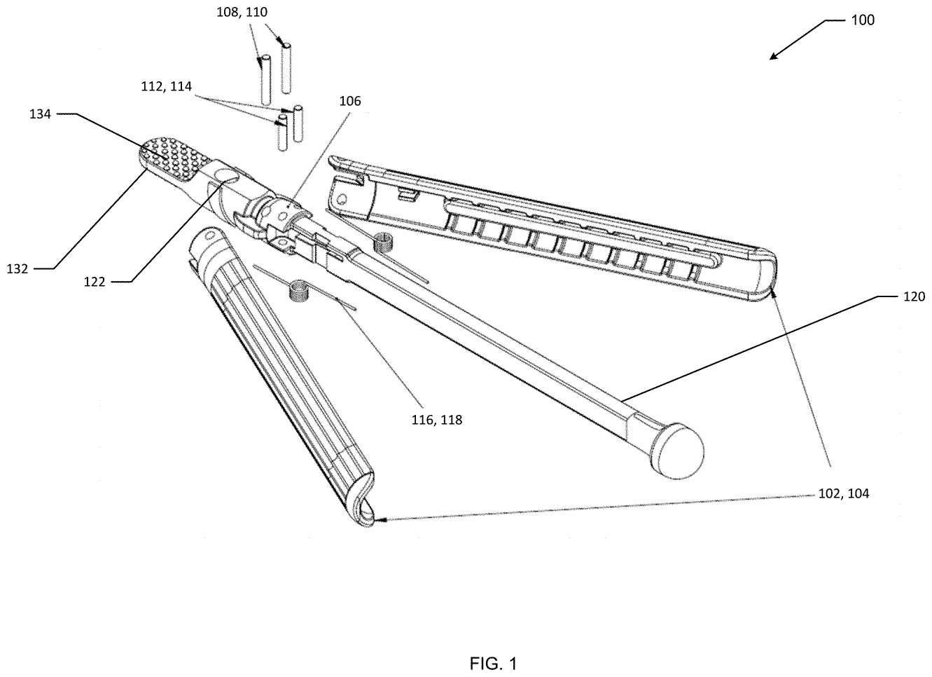

Referring now to the drawings, FIG. 1 illustrates an organ retractor 100 in an open configuration. The organ retractor 100 includes at least two retractor jaws, such as a first retractor jaw 102 and a second retractor jaw 104. In an example embodiment, first retractor jaw 102 and second retractor jaw 104 are interchangeable, but for their orientation with respect to organ retractor 100. Each of the first retractor jaw 102 and the second retractor jaw 104 is hingedly connected to a central housing 106 of the organ retractor 100. Namely, the first retractor jaw 102 and the second retractor jaw 104 are hingedly connected to the central housing 106 via a plurality of jaw hinge pins 108, 110. For example, each of the first retractor jaw 102 and the second retractor jaw 104 pivot about one of the jaw hinge pins 108, 110.

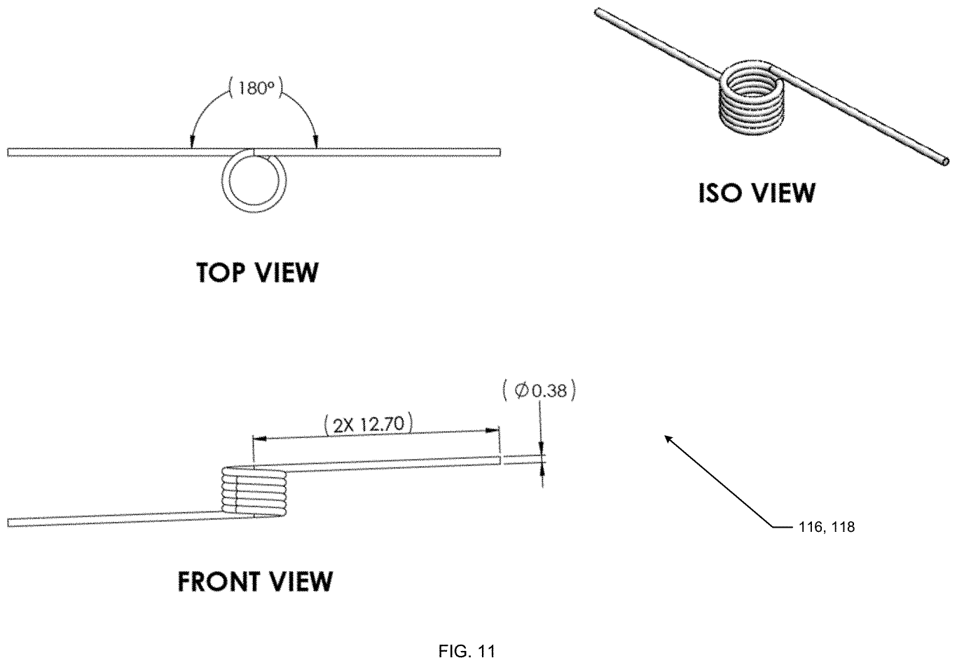

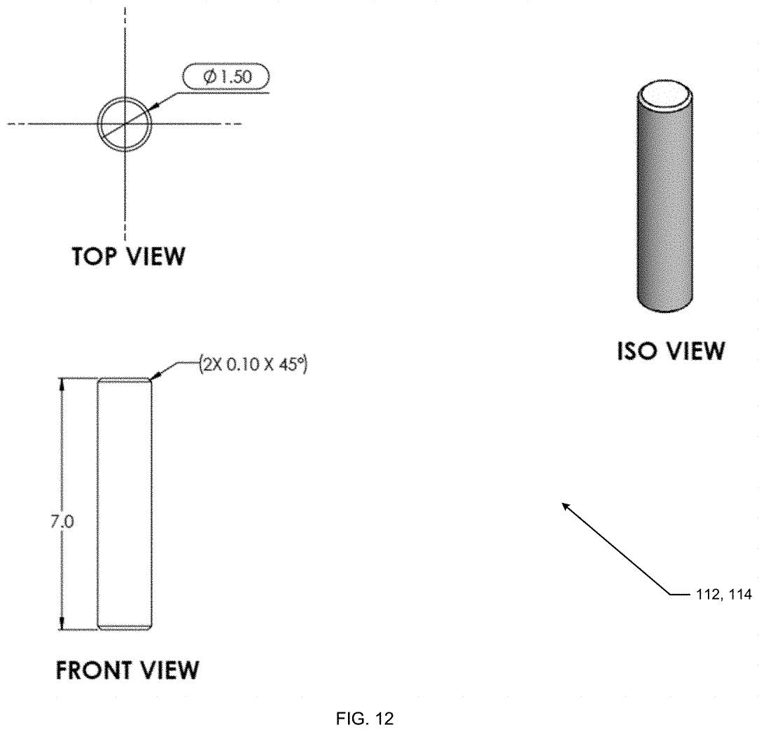

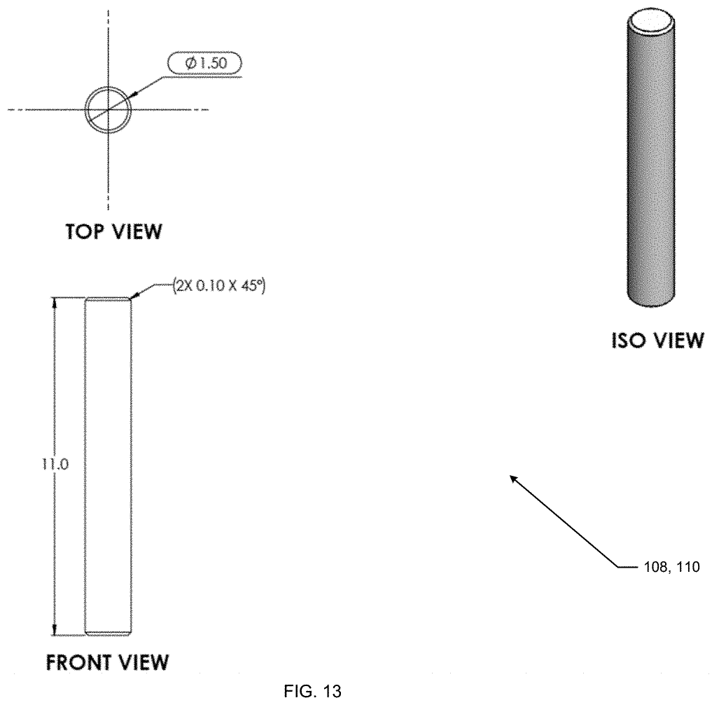

The central housing 106 also includes one or more torsion springs 116, 118 connected to the central housing 106 via spring pins 112, 114. For example, torsion spring 116 is coupled to central housing 106 via spring pin 112, such that spring pin 112 passes through the center loop of torsion spring 116. Similarly, for example, torsion spring 118 is coupled to central housing 106 via spring pin 114, such that spring pin 114 passes through the center loop of torsion spring 118. A torsion spring 116, 118 engages with each of the first retractor jaw 102 and the second retractor jaw 104. For example, an offshoot from the torsion spring 116 engages with the first retractor jaw 102 and the central housing 106; likewise, an offshoot from the torsion spring 118 engages with the second retractor jaw 104 and the central housing 106. Specific dimensioning for each of torsion springs 116, 118, spring pins 112, 114, and jaw hinge pins 108, 110 is generally illustrated by FIGS. 11 to 13, respectively. That said, it should be appreciated that these dimensions are exemplary only, and other similar dimensioning is contemplated herein.

Generally, these torsion springs 116, 118 urge the first retractor jaw 102 and the second retractor jaw 104 away from each other, such that the organ retractor 100 is generally disposed or biased in the open configuration by the torsion springs 116, 118. In the open configuration, the first retractor jaw 102 and the second retractor jaw 104 generally form a v-shape.

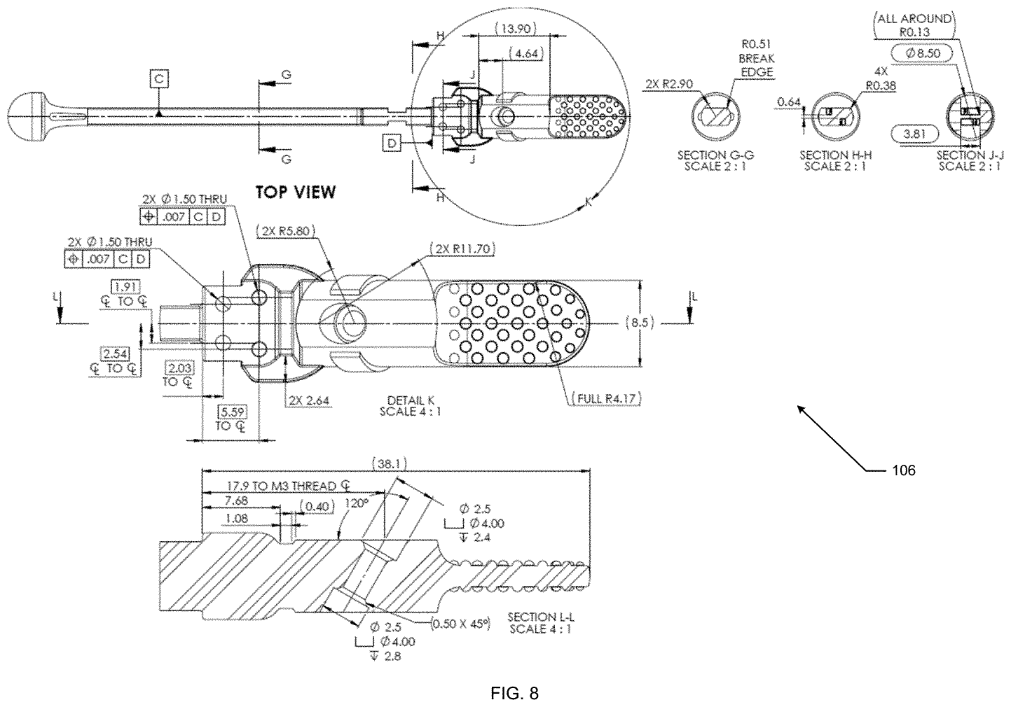

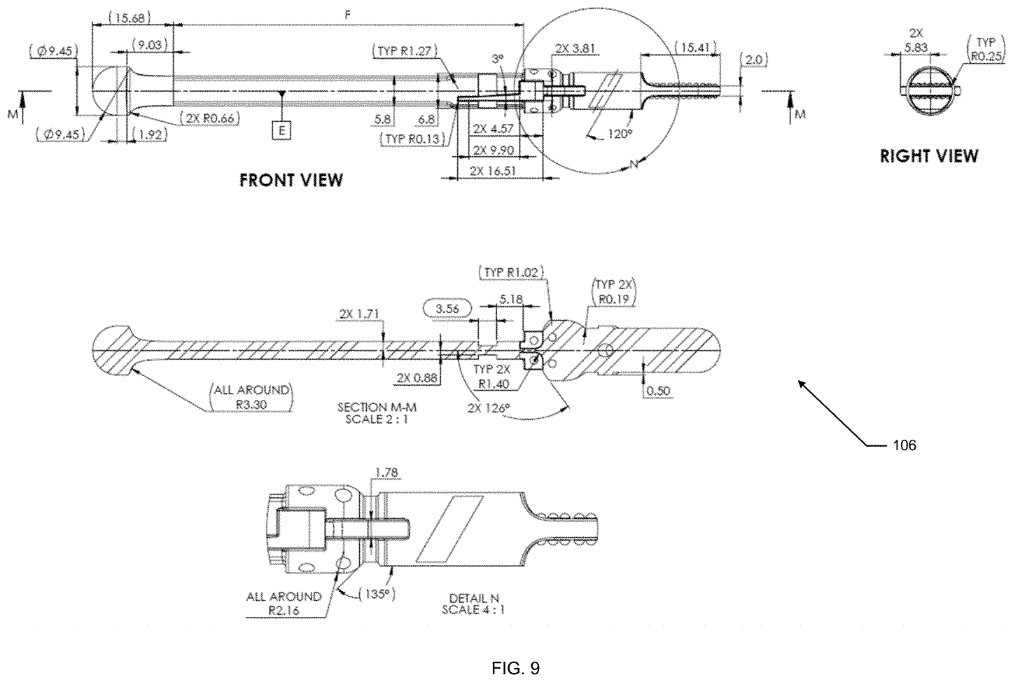

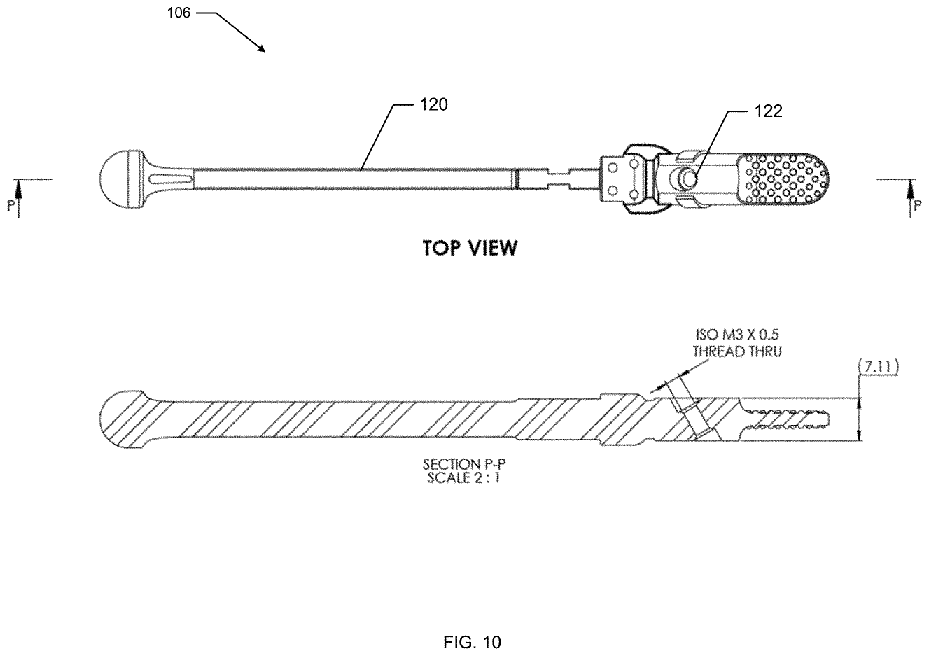

The organ retractor 100 further includes a central support structure 120. In an embodiment, the central support structure 120 is coupled directly to the central housing 106. In a different embodiment, the central support structure 120 is formed integrally with the central housing 106. For example, housing 106 and central support structure 120 could be one single piece of injection-molded material. When the organ retractor 100 is in the open configuration, the central support structure 120 is disposed generally between the first retractor jaw 102 and the second retractor jaw 104. For example, an angle between the first retractor jaw 102 and the central support structure 120 is approximately equal to an angle between the second retractor jaw 104 and the central support structure 120. In an embodiment, the central support structure 120 is sized and shaped to engage with a portion of a liver. The central support structure 120 may include surface-contouring, and related features, along with a blunt distal end (e.g., mushroom-shaped end) to reduce or eliminate any undesirable trauma to organs. Specific structure of central support structure 120 and central housing 106, for example, is illustrated generally by FIGS. 8 to 10 herein.

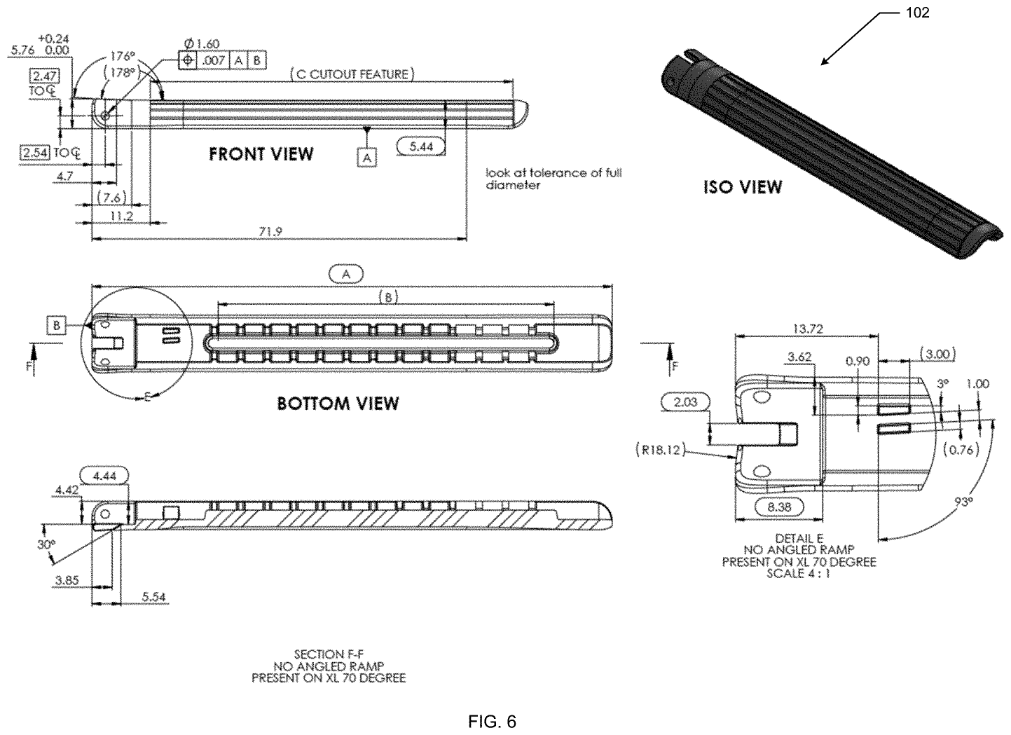

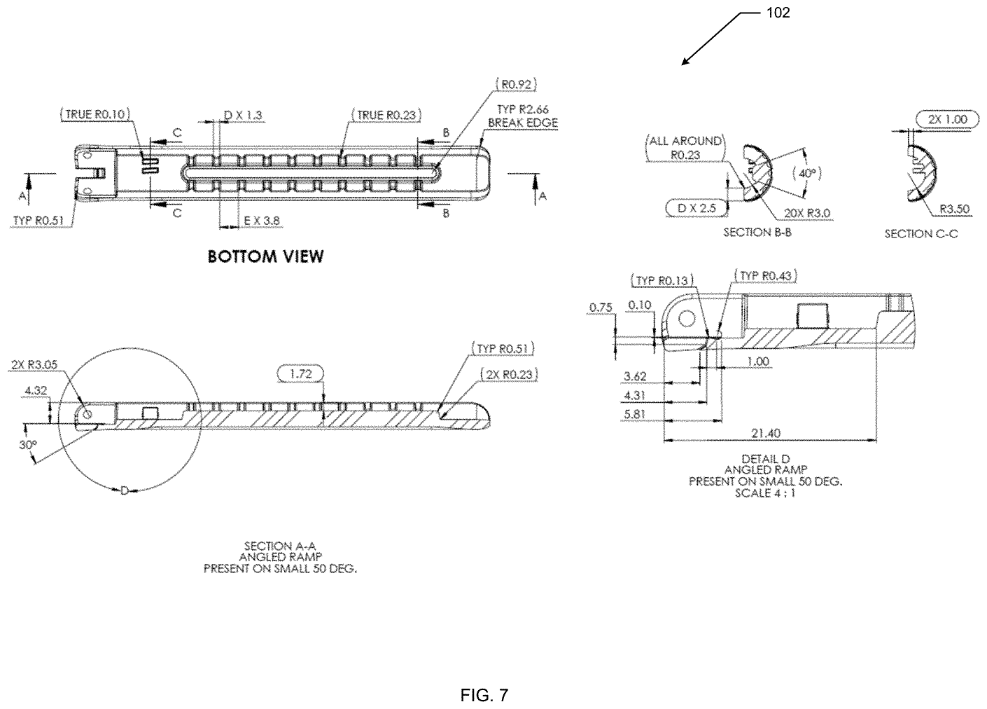

The central support structure 120 is generally configured to be disposed between the first retractor jaw 102 and the second retractor jaw 104; namely, when the organ retractor 100 is in a closed configuration (prior to insertion, for example), the central support structure 120 is at least partially disposed within the first retractor jaw 102 and the second retractor jaw 104. For example, each of the first retractor jaw 102 and the second retractor jaw 104 may include recesses, configured to receive a portion of the central support structure 120 when the organ retractor 100 is in a closed configuration. Specific structure of retractor jaw 102, for example, is illustrated generally by FIGS. 6 and 7 herein.

The central housing 106 of the organ retractor 100 includes a threaded portion 122, such as a threaded hole or aperture. The threaded portion 122 is disposed on the organ retractor 100 proximate to the jaw hinge pins 108, 110. In an embodiment, the threaded portion 122 is disposed along an axis that is perpendicular to a plane defined by the central support structure 120 and the retractor jaws 102, 104, when the organ retractor 100 is in the open configuration. In a different embodiment, the threaded portion 122 is disposed along an axis that angled relative to the plane defined by the central support structure 120 and the retractor jaws 102, 104, when the organ retractor 100 is in the open configuration.

The central housing 106 of the organ retractor 100 may further include an end piece 132. In an embodiment, the end piece 132 may extend distally from the threaded portion 122. The end piece 132 may be generally rectangular in shape, although other shapes are contemplated. The end piece 132 may include a plurality of ridges 134, or other surface features. For example, a surgeon may grab the organ retractor 100 at the end piece 132, and subsequently move, positon, or remove the organ retractor 100, via a surgical grasping instrument, as described in greater detail herein.

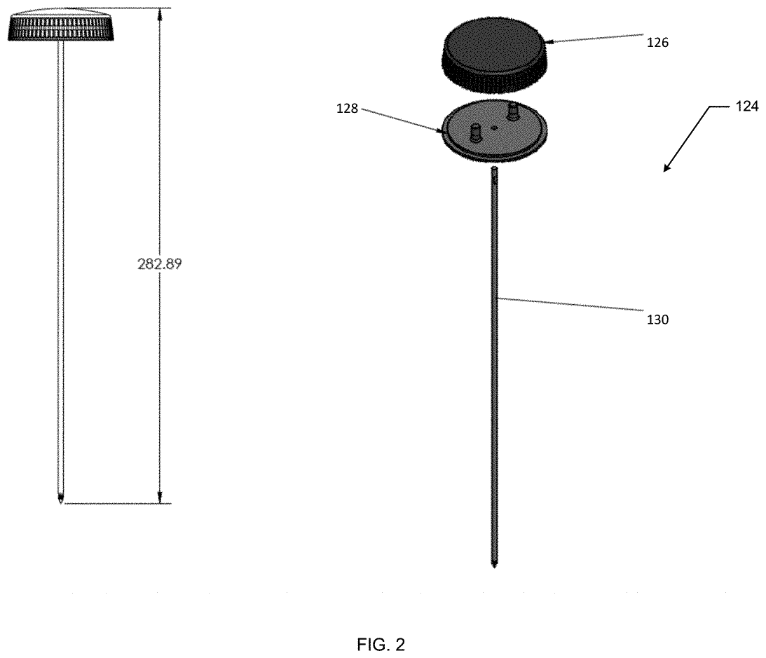

With reference to FIGS. 1 and 2, the threaded portion 122 is generally configured to engage with a threaded obturator 124. For example, a surgeon can rotate the threaded obturator 124 to engage with the threaded portion 122 of organ retractor 100, and subsequently manipulate the organ retractor 100 via the threaded obturator 124, as described in greater detail herein. In an embodiment, threaded obturator 124 includes a cap 126 coupled to a base 128. The cap may include ridges or other surface features to improve grip, such as during rotation of the threaded obturator 124. The threaded obturator further includes a shaft 130 with a threaded distal end. For example, the distal end of shaft 130 is configured to engage with the threaded portion 122 of organ retractor 100.

In light of the disclosure above regarding the structural components of the organ retractor 100, it is helpful to explain how the organ retractor is used in the surgical environment.

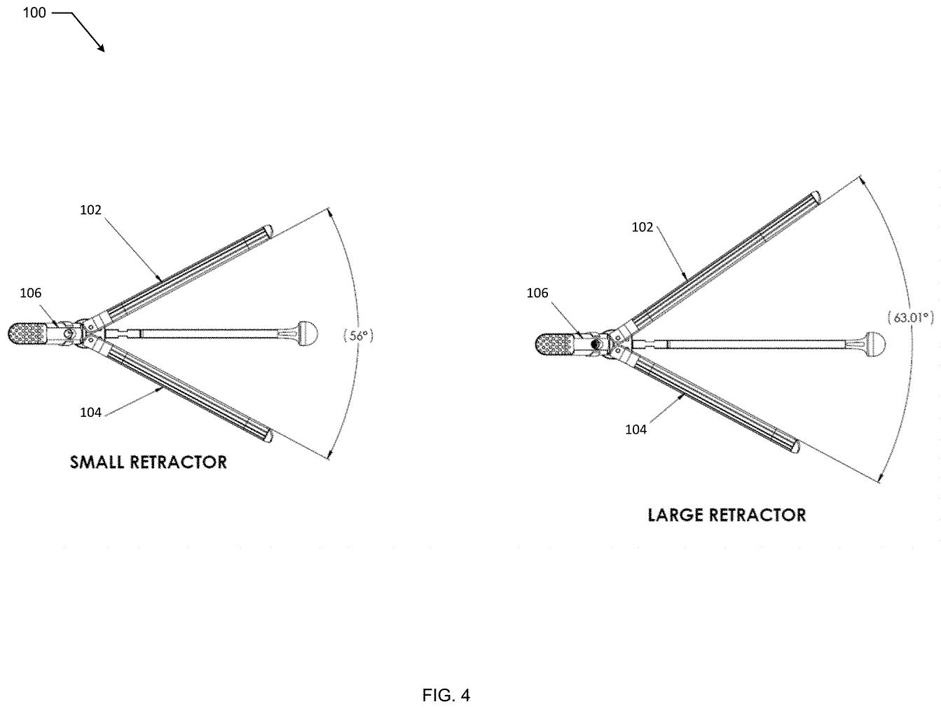

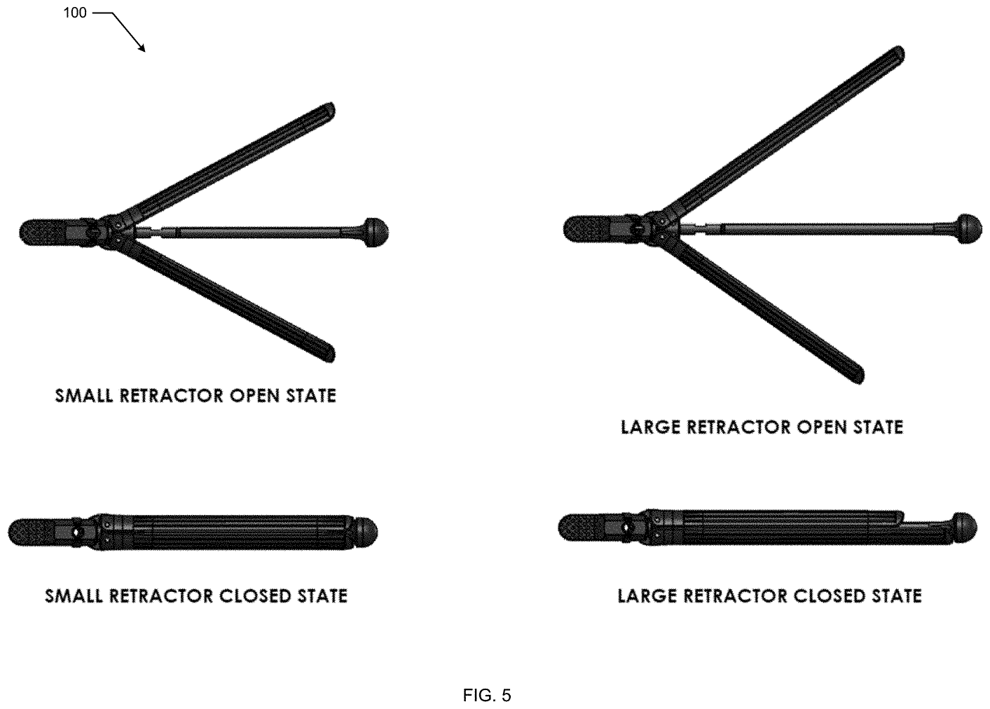

In typical gastrointestinal surgeries, a 12 mm trocar is implanted onto the patient's abdomen, to provide the surgeon with a window of access into the patient's body cavity. The surgeon will manually collapse the first retractor jaw 102 and the second retractor jaw 104 together (against the spring force of the torsion springs 116, 118), and subsequently insert the organ retractor 100 through the 12 mm trocar. In a preferred embodiment, the organ retractor 100 is inserted such that the end piece 132 is the last component to enter the 12 mm trocar. Once the organ retractor 100 passes through the 12 mm trocar, and enters the patient's body cavity, the organ retractor 100 will spring into the open configuration. Namely, each of the first retractor jaw 102 and second retractor jaw 104 are urged away from each other by the torsion springs 116, 118, thus exposing the central support structure 120. The open and closed configurations of various organ retractors 100 (including different sized retractors) are illustrated generally by FIGS. 4 and 5 herein. Namely, in closed configurations, organ retractors 100 have a small profile, for ready insertion through a 12 mm trocar.

The surgeon then passes the threaded obturator 124 through a small incision on the patient. In an embodiment, the incision is a 2 mm incision. Because only the threaded obturator 124 needs to pass through this incision (as opposed to a 5 mm hook or other support structure), the incision size is minimized.

Via cameras or other optical techniques, the surgeon locates the threaded portion 122 of the organ retractor 100, and subsequently engages the threaded obturator 124 with the threaded portion 122 of organ retractor 100 by twisting the cap 126 and base 128 of the threaded obturator 124. Once the threaded obturator 124 is engaged with the organ retractor 100, the surgeon may position the organ retractor 100 against or underneath an organ, such as the liver. The surgeon then “lifts” or “pulls” on the threaded obturator 124, such that the central support structure 120 along with the retractor jaws 102, 104 retract the organ. For example, the surgeon positions the organ retractor 100 under the liver and lifts up, such that the liver is moved up and out of the way of the stomach by central support structure 120 and retractor jaws 102, 104.

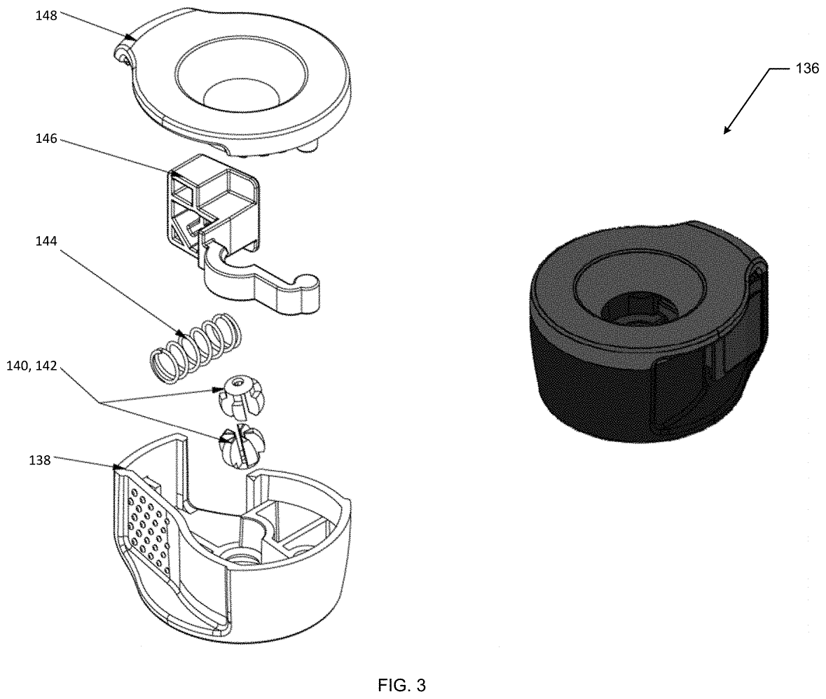

The threaded obturator 124 is then clamped in place outside of the patient's body cavity. For example, with reference to FIG. 3, a clamp housing 136 is generally disposed along the shaft 130 of the threaded obturator 124 near the exterior surface of the patient's skin. The clamp housing 136 includes a base 138 with an aperture, such that the shaft 130 of the threaded obturator 124 extends through the base 138. The clamp housing 136 includes two clamp balls 140, 142 aligned with one another and further aligned with the aperture of base 138. For example, the clamp balls 140, 142 are selectively deformable (as disclosed herein). When clamp balls 140, 142 are not deformed, shaft 130 of threaded obturator 124 slides easily within clamp balls 140, 142. When clamp balls 140, 142 are selectively deformed, shaft 130 of threaded obturator 124 is “locked” by clamp balls 140, 142, such that shaft 130 cannot slide relative to clamp balls 140, 142.

Continuing on, clamp housing 136 includes spring 144 configured to engage with lever 146. Namely, lever 146 is disposed in an “open” positon via spring 144; lever 146 may be moved to a “closed” position against the force of spring 144. Clamp housing 136 further includes a cap 148 to ensure that clamp balls 140, 142, spring 144, and lever 146 remain disposed in clamp housing 136 between cap 148 and base 138.

As noted prior, in the unlocked position, the shaft 130 of the threaded obturator 124 slides within clamp housing 136. For example, once the threaded obturator 124 is lifted (along with the organ to be moved during surgery), such that the organ is retracted by central support structure 120 and retractor jaws 102, 104, the clamp housing 136 is slid adjacent to the patient's skin and “locked” into place, such that base 138 contacts the skin of the patient. The lever 146 is moved to a “closed” position, such that it deforms the clamp balls 140, 142, thus locking the threaded obturator 124 relative to the clamp housing 136 (and thus relative to the patient's skin).

This may advantageously ensure that the shaft 130 of the threaded obturator 124 remains fixed and does not slide relative to the patient's skin. Thus, the entire organ retractor 100 remains fixed relative to the patient's skin, such that the retracted organ is held “up” or “away” via central support structure 120 and retractor jaws 102, 104. By implementing clamp housing 136 with threaded obturator 124, the organ retractor 100 avoids the need for any additional physical mounts or support arms that are commonplace with Nathanson liver retractor systems.

Upon completion of the surgical procedure, once surgical access is no longer necessary, the clamp housing 136 is unlocked from the shaft 130 of the threaded obturator 124. The surgeon then disengages the threaded obturator 124 from the organ retractor 100 by un-twisting the cap 126 and base 124 of the threaded obturator 124. The threaded obturator 124 is removed from the surgical environment (via the small incision). The surgeon then grasps the organ retractor 100 via a surgical grasping tool. Specifically, for example, the surgeon passes a surgical grasping tool through the 12 mm trocar; the surgeon grasps the end piece 132 of the organ retractor 100 with the surgical grasping tool. Once the organ retractor 100 is grasped, the surgeon can “pull” the organ retractor 100 out of the patient's body cavity through the 12 mm trocar. Due to the hinged orientation of the first retractor jaw 102 and the second retractor jaw 104, the 12 mm trocar will “close” each of the first retractor jaw 102 and the second retractor jaw 104 as the organ retractor 100 is pulled through the 12 mm trocar by the end piece 132.

This organ retractor 100 and related procedures disclosed herein advantageously eliminate additional external support structures, thus improving setup time and simplifying the retraction procedure. The organ retractor 100 and related procedures utilize existing trocars and eliminate the need for a larger 5 mm incision, thus reducing the total number of incisions and improving patient outcomes. The organ retractor 100 and related procedures retract organs over a distributed surface including the central support structure 120, thus reducing risk of patient trauma such as hematoma. The organ retractor and related procedures are generally easier to manipulate, affix, and remove.

As used in this specification, including the claims, the term “and/or” is a conjunction that is either inclusive or exclusive. Accordingly, the term “and/or” either signifies the presence of two or more things in a group or signifies that one selection may be made from a group of alternatives.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated. Note that elements recited in means-plus-function format are intended to be construed in accordance with 35 U.S.C. § 112 ¶6. The scope of the invention is therefore defined by the following claims.

The invention is claimed as follows:

1. An organ retractor comprising:
a plurality of retractor jaws;
a central support structure; and
a central housing including a threaded portion,
wherein the plurality of retractor jaws is hingedly connected to the central housing,
wherein the central support structure is coupled to the central housing, and
wherein the plurality of retractor jaws is disposed in an open position by a spring.

2. The organ retractor of claim 1, wherein the central support structure is disposed between the plurality of retractor jaws.

3. The organ retractor of claim 1, wherein the plurality of retractor jaws includes two retractor jaws.

4. The organ retractor of claim 1, further comprising a threaded obturator, configured to engage with the threaded portion of the central housing.

5. The organ retractor of claim 4, wherein, responsive to engaging the threaded obturator with the threaded portion of the central housing, the organ retractor is capable of being manipulated via the threaded obturator.

6. The organ retractor of claim 5, wherein manipulation includes lifting the threaded obturator, such that the organ retractor is lifted to support an organ.

7. The organ retractor of claim 6, wherein the organ is generally supported by the plurality of retractor jaws and the central support structure.

8. The organ retractor of claim 5, wherein, responsive to lifting the threaded obturator, the threaded obturator is clamped at an exterior of a patient, such that the threaded obturator is prevented from sliding within an incision on the patient.

9. The organ retractor of claim 1, wherein the central housing includes an end piece, configured to be manipulated by a surgical grasping tool.

10. The organ retractor of claim 1, wherein the plurality of retractor jaws, the central support structure, and central housing are configured to be inserted, into a patient, via a port.

11. The organ retractor of claim 10, wherein the plurality of retractor jaws are inserted into the patient in a closed configuration, such that the plurality of retractor jaws are closed around the central support structure.

12. The organ retractor of claim 11, wherein, responsive to exiting the port and entering the patient, the plurality of retractor jaws are disposed into the open position via the spring, away from the central support structure.

13. An organ retractor comprising:
two retractor jaws, hingedly coupled to one another; and
a central support structure disposed between the retractor jaws,
wherein the retractor jaws are disposed in an open position by a spring.

14. The organ retractor of claim 13, wherein the central support structure is sized and shaped to retract a liver.

15. The organ retractor of claim 13, further comprising a central housing including a threaded portion, wherein the retractor jaws are hingedly connected to the central housing, and wherein the central support structure is coupled to the central housing.

16. The organ retractor of claim 15, further comprising a threaded obturator, configured to engage with the threaded portion of the central housing.

17. The organ retractor of claim 16, wherein, responsive to engaging the threaded obturator with the threaded portion of the central housing, the organ retractor is capable of being manipulated via the threaded obturator.

18. The organ retractor of claim 17, wherein manipulation includes lifting the threaded obturator, such that the organ retractor is lifted to support an organ.

19. The organ retractor of claim 18, wherein, responsive to lifting the threaded obturator, the threaded obturator is clamped at an exterior of a patient, such that the threaded obturator is prevented from sliding within an incision on the patient.

20. The organ retractor of claim 15, wherein the central housing includes an end piece, configured to be manipulated by a surgical grasping tool.
